(19) United States
(12) Patent Application Publication
(10) Pub. No.: US 2017/0258625 A1
(43) Pub. Date: Sep. 14, 2017

(54) METATARSAL PAIN RELIEF GEL STRIP

(71) Applicant: Inwest Manufacturing Lp, Riverton, UT (US)

(72) Inventor: Jeffrey W. Lavery, Draper, UT (US)

(21) Appl. No.: 15/068,180

(22) Filed: Mar. 11, 2016

(51) Int. Cl. A61F 5/30; A61F 5/01

(52) U.S. Cl. CPC A61F 5/30; A61F 5/019; A61F 5/14

(57) ABSTRACT

A metatarsal pain relief gel strip can be used to treat and/or prevent Morton's neuroma and possibly other forefoot conditions. The pain relief gel strip can include an arched or protruding gel structure that is secured within an adhesive material to allow the gel structure to be maintained on the underside of the foot between adjacent toes. Due to the arched shaped of the gel structure, when the wearer places weight on the foot, the gel structure will apply a separating force on the toes thereby relieving pressure on the nerve that runs between the toes. In this way, the pain relief gel strip can minimize the likelihood of developing Morton's neuroma, minimize pain and discomfort when Morton's neuroma has already occurred, or assist in minimizing the swelling and inflammation of the nerve.

### CROSS-REFERENCE TO RELATED APPLICATIONS

N/A

### BACKGROUND

A neuroma is a thickening of nerve tissue that may develop in various parts of the body. The most common neuroma in the foot is Morton's neuroma, which most commonly occurs between the third and fourth toes. It is sometimes referred to as an intermetatarsal neuroma. “Intermetatarsal” describes its location in the ball of the foot between the metatarsal bones. Neuromas may also occur in other locations in the foot. The thickening, or enlargement, of the nerve that defines a neuroma is the result of compression and irritation of the nerve. This compression creates enlargement of the nerve, eventually leading to permanent nerve damage.

FIG. 1A illustrates a foot 100 when the nerve 101 between the third and fourth toes is in a normal, non-inflamed condition. In contrast, FIG. 1B illustrates how nerve 101 can become inflamed thereby forming a neuroma 101a. As shown, Morton's neuroma typically occurs as the nerve passes under the ligament connecting the metatarsals in the forefoot.

Anything that causes compression or irritation of the nerve can lead to the development of a neuroma. For example, any sporting activity that involves repeated use of the forefoot, such as running or cycling, can cause Morton's neuroma in some individuals. The pain associated with Morton's neuroma can range from bothersome to debilitating.

### BRIEF SUMMARY

The present invention is directed to a metatarsal pain relief gel strip that can be used to treat and/or prevent Morton's neuroma and possibly other forefoot conditions. The pain relief gel strip of the present invention can include an arched or protruding gel structure that is secured within an adhesive material to allow the gel structure to be maintained on the underside of the foot between adjacent metatarsals. Due to the arched shaped of the gel structure, when the wearer places weight on the foot, the gel structure will apply a separating force on the toes thereby relieving pressure on the nerve that runs between the metatarsals. In this way, the pain relief gel strip can minimize the likelihood of developing Morton's neuroma, minimize pain and discomfort when Morton's neuroma has already occurred, or assist in minimizing the swelling and inflammation of the nerve.

In one embodiment, the present invention is implemented as a pain relief gel strip that comprises: a gel structure having a thickness, a length, and a width; and an adhesive material having a length and a width that are greater than the length and the width of the gel structure. The gel structure is secured within the length and the width of the adhesive material such that the adhesive material can be employed to secure the gel structure to the underside of the foot.

In another embodiment, the present invention is implemented as a pain relief gel strip comprising: a gel structure having a thickness greater than 0.10 inches, a length, and a width; and an adhesive material having a length and a width that are greater than the length and the width of the gel structure. The gel structure is secured within the length and the width of the adhesive material such that the adhesive material can be employed to secure the gel structure to the underside of the forefoot between adjacent metatarsals. The thickness and the width of the gel structure causes a separating force to be applied on the adjacent metatarsals when weight is placed on the forefoot.

In another embodiment, the present invention is implemented as a metatarsal pain relief gel strip comprising: a generally flat adhesive material having a width and a height, a top side of the adhesive material including an adhesive; and a gel structure having a thickness, a width, and a height, the width and the height of the gel structure being less than the width and the height of the adhesive material, the gel structure having a generally flat bottom side that is secured to the top side of the adhesive material and a top side that protrudes upwardly away from the top side of the adhesive material, the top side of the gel structure having an arched profile. The top side of the adhesive material is configured to be secured to the underside of the forefoot to thereby maintain the gel structure between adjacent metatarsals with the top side of the gel structure protruding between the metatarsals to thereby apply a separating force against the metatarsals when weight is placed on the forefoot.

This summary is provided to introduce a selection of concepts in a simplified form that are further described below in the Detailed Description. This Summary is not intended to identify key features or essential features of the claimed subject matter.

### BRIEF DESCRIPTION OF THE DRAWINGS

Understanding that these drawings depict only typical embodiments of the invention and are not therefore to be considered limiting of its scope, the invention will be described and explained with additional specificity and detail through the use of the accompanying drawings in which:

FIG. 1A illustrates a normal condition of a nerve that runs between the toes;

FIG. 1B illustrates the nerve of FIG. 1A when Morton's neuroma has occurred;

FIG. 2 illustrates a top perspective view of a metatarsal pain relief gel strip that is configured in accordance with one or more embodiments of the present invention;

FIG. 2A illustrates a side view of the metatarsal pain relief gel strip of FIG. 2;

FIG. 2B illustrates an exploded side view of the metatarsal pain relief gel strip of FIG. 2;

FIG. 2C illustrates a bottom view of the metatarsal pain relief gel strip of FIG. 2;

FIG. 2D illustrates a top view of the metatarsal pain relief gel strip of FIG. 2;

FIG. 3 illustrates a top perspective view of a metatarsal pain relief gel strip that is configured in accordance with one or more other embodiments of the present invention;

FIG. 3A illustrates a side view of the metatarsal pain relief gel strip of FIG. 3;

FIG. 3B illustrates an exploded side view of the metatarsal pain relief gel strip of FIG. 3;

FIG. 3C illustrates a bottom view of the metatarsal pain relief gel strip of FIG. 3;

FIG. 3D illustrates a top view of the metatarsal pain relief gel strip of FIG. 3;

FIGS. 4A-4C illustrate suitable dimensions for the metatarsal pain relief gel strips of FIGS. 2 and 3; and

FIG. 5 illustrates how the metatarsal pain relief gel strips of FIGS. 2 and 3 can be applied to the bottom of the forefoot.

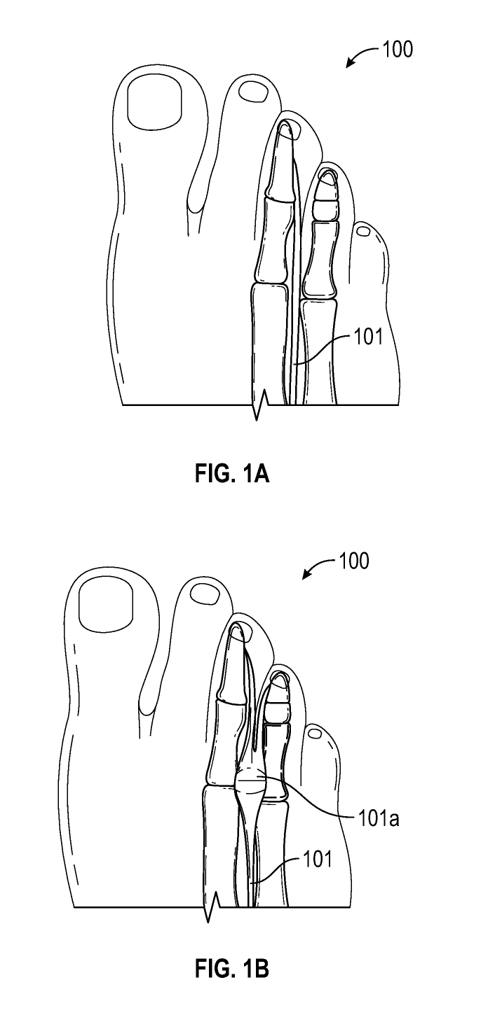

### DETAILED DESCRIPTION

In this specification, the top surface should be construed as the surface of the metatarsal pain relief gel strip that will be adhered to the underside of the forefoot. In other words, the top surface of the strip will face upward when the strip is properly worn on the foot.

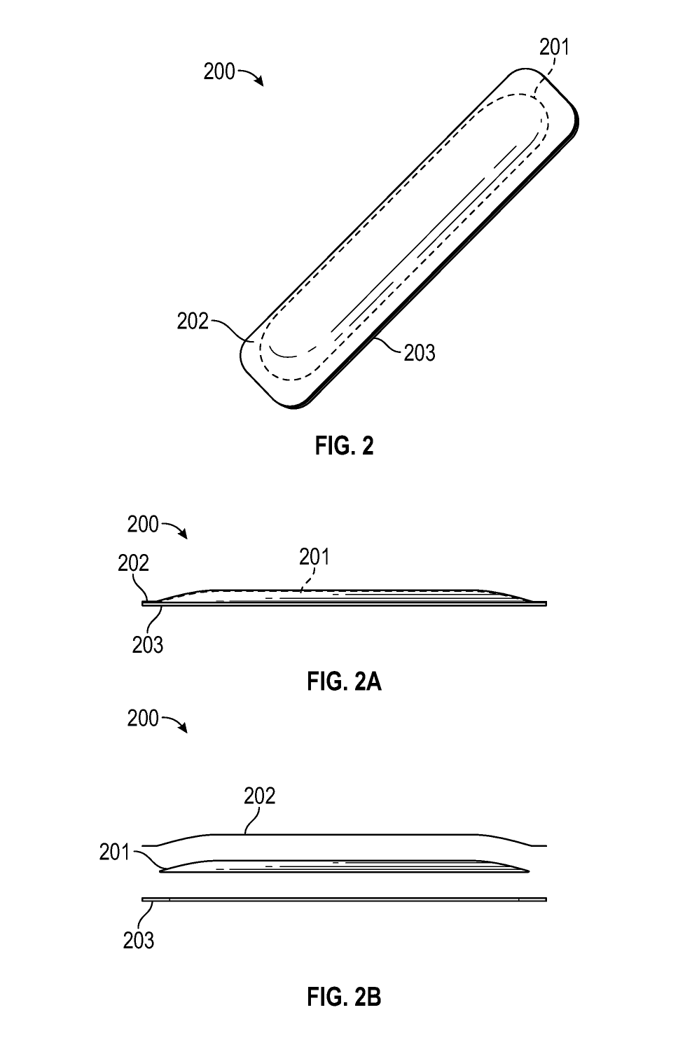

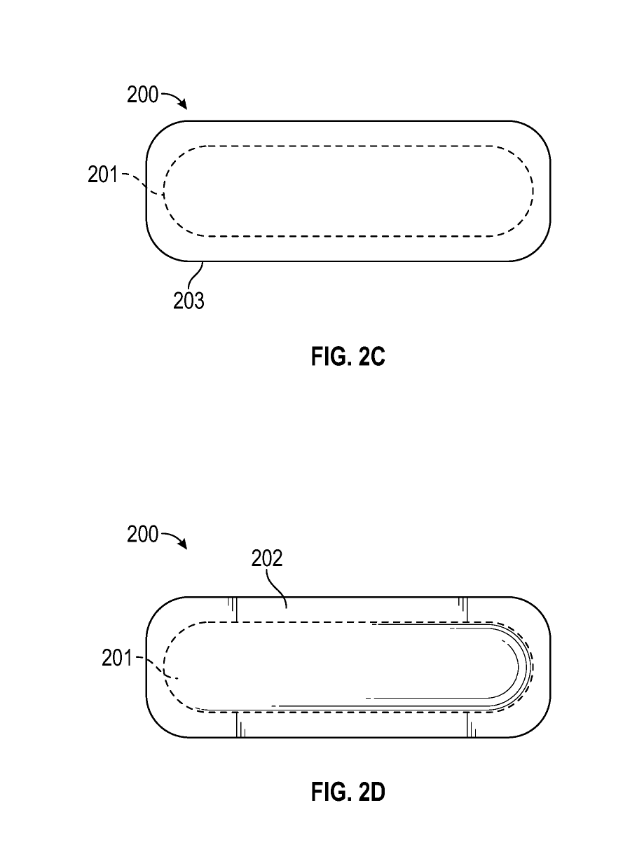

FIGS. 2 and 2A-2D each illustrate a different view of a metatarsal pain relief gel strip 200 that is configured in accordance with one or more embodiments of the present invention. Strip 200 includes an arched or protruding gel structure 201 that is secured within (or sandwiched between) a top adhesive material 202 and a bottom adhesive material 203. Although not shown, a backing can also be secured to a top side of adhesive material 202. This backing can be configured to peel away from adhesive material 202 to allow strip 200 to be secured to the underside of the forefoot.

Adhesive material 203 can include a single side (i.e., a top side) that forms an adhesive surface that can adhere to a bottom side of adhesive material 202 (which bottom side in some embodiments may also include an adhesive). The adhesion between the top side of adhesive material 203 and the bottom side of adhesive material 202 secures gel structure 201 within the adhesive materials. Further, the top side of adhesive material 202 may form an adhesive surface to allow strip 200 to be secured to the skin.

As best seen in FIGS. 2A and 2B, which provide a side view and an exploded side view respectively of strip 200, gel structure 201 has an arched shape so that it will protrude upwardly from adhesive material 203. Gel structure 201 and adhesive materials 202 and 203 can be formed of different materials. For example, gel structure 201 may be formed of silicone or another similar flexible and compressible polymer while adhesive materials 202 and 203 can be formed of any medical grade adhesive tape or material similar to that used for adhesive bandages. Gel structure 201 can be centered within adhesive materials 202 and 203 (i.e., adhesive materials 202 and 203 can be wider and longer than gel structure 201) so that the adhesive material can be secured to the bottom of the forefoot to maintain the position of gel structure 201.

Alternatively, in some embodiments, gel structure 201 and adhesive materials 202 and 203 can be formed of the same material. For example, a unitary component having the combined shape of gel structure 201 and adhesive materials 202 and 203 could be molded from silicone or similar material. In such cases, an adhesive could be applied to the top surface of the unitary component to allow it to be secured to the underside of the forefoot. However, due to the difficultly of applying an adhesive to silicone or other similar polymers, the sandwich configuration shown in the figures may be preferred. This sandwich configuration will also allow gel structure 201 to move somewhat within adhesive material 202 and 203 to facilitate adjustments in the placement of gel structure 201 even after adhesive material 202 has been secured to the skin.

As is also best shown in FIGS. 2A and 2B, gel structure 201 can have a generally flat bottom surface (i.e., the surface that faces adhesive material 203) and an arched or protruding top surface (i.e., the surface that faces adhesive material 202 and will therefore be oriented towards the skin). This arched profile will cause strip 200 to apply a separating force to adjacent toes when the strip is worn on the underside of the foot. More specifically, strip 200 can be positioned between the metatarsals so that when weight is placed on the foot (e.g., while walking, running, or performing another activity), gel structure 201 will be forced upwardly between the metatarsals. The arched or rounded top surface of gel structure 201 will allow an upward and outward force to be applied to the metatarsals without undue discomfort. Forming gel structure 201 of silicone or of another relatively flexible and compressible polymer material can further ensure that undue discomfort will not be caused.

By forming gel structure 201 with a generally flat bottom surface and an arched top surface, strip 200 can be worn with minimal impact on the wearer. For example, because the bottom surface is flat and because it is formed of a relatively flexible and compressible material, the bottom surface of gel structure 201 will substantially conform to the contour of the underside of the forefoot when stepped on. This will minimize the feeling that something is being stepped on. However, due to the upwardly protruding configuration, the top surface of gel structure 201 will still provide the necessary separation force on the adjacent metatarsals. In essence, strip 200 can be worn in a similar manner as an adhesive bandage is worn while also providing a way to treat and/or prevent Morton's neuroma.

As best shown in FIGS. 2C and 2D, gel structure 201 can have a generally rounded rectangular shape. The rectangular shape ensures that strip 200 will have sufficient length to provide a separating force along a substantial length of the adjacent metatarsals. By spreading the force along the length of the metatarsals, greater comfort is also obtained. In particular, the rectangular shape can minimize the “rock-in-the-shoe” feeling since the separating force will be spread lengthwise along the metatarsals rather than focused on a particular point.

FIGS. 2C and 2D also illustrate how gel structure 201 can be centered within adhesive materials 202 and 203 so that a portion of adhesive materials 202 and 203 extends around the entire perimeter of gel structure 201. In this way, adhesive materials 202 and 203 can form a pocket within which gel structure 201 is maintained. In some embodiments, adhesive material 203 can have a bottom side/surface that is smooth to minimize the likelihood that a sock or any other surface that the foot comes in contact with will catch on the adhesive material to cause it to peel off prematurely.

In contrast to what is shown in the figures, in some embodiments, a pain relief gel strip may not include a top adhesive material. For example, if gel structure 201 is formed of a material to which adhesive material 203 may readily adhere, adhesive material 202 may not be required to secure gel structure 201 within the pain relief gel strip. Instead, the top side of adhesive material 203 could be applied directly to the skin to maintain gel structure 201 in position. However, because gel structure 201 may preferably be formed of silicone, and because many suitable adhesives do not readily adhere to silicone, top adhesive material 202 can be employed to sandwich and secure gel structure 201 within strip 200.

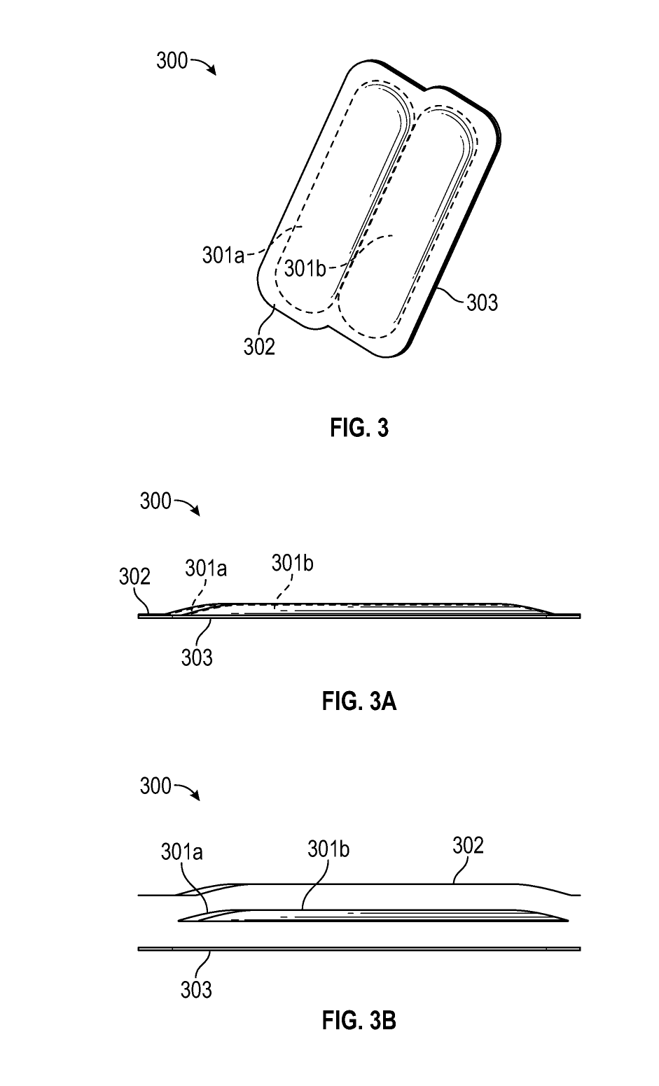

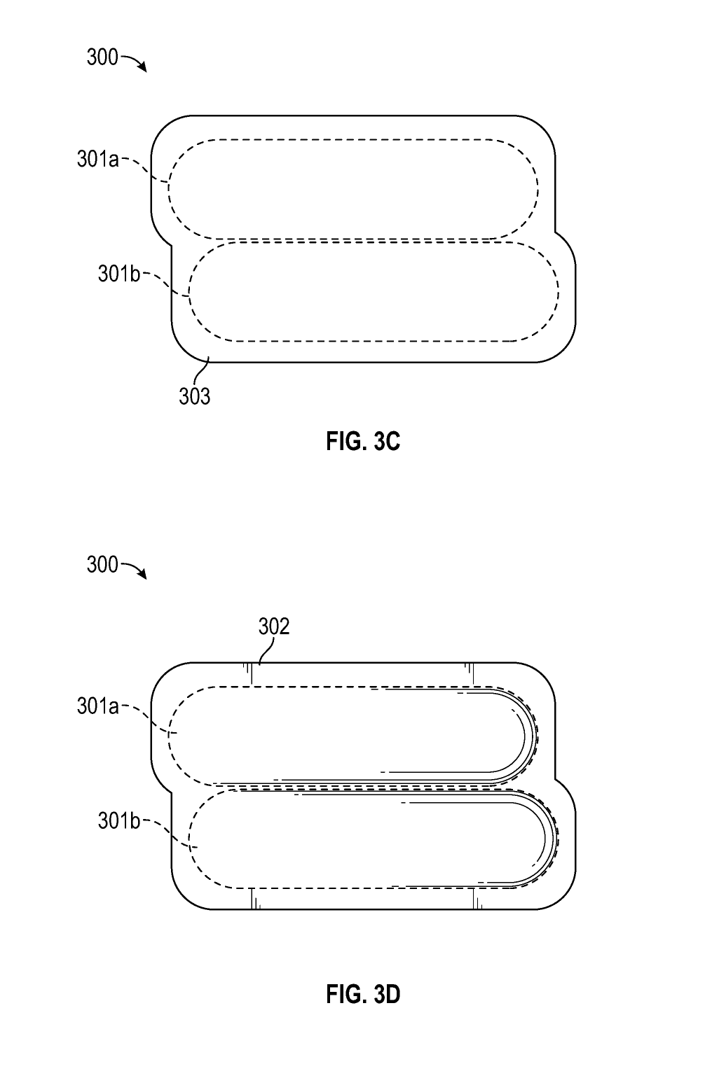

FIGS. 3 and 3A-3D each illustrate a different view of another metatarsal pain relief gel strip 300 that is configured in accordance with one or more embodiments of the present invention. Strip 300 is substantially similar to strip 200 except that strip 300 includes two gel structures 301a, 301b. Gel structures 301a, 301b can be positioned substantially in parallel so that they may be positioned between adjacent sets of metatarsals. Because of the arched shape of gel structures 301a, 301b, a gap will exist between the structures even when they are side-by-side as shown in FIG. 3. This gap can be positioned directly beneath one of the metatarsals so that gel structures 301a and 301b will be positioned between adjacent metatarsals. In other embodiments, gel structures 301a and 301b may be slightly spaced from one another but may still extend in parallel.

Also, as best shown in FIGS. 3C and 3D, gel structures 301a, 301b may be slightly lengthwise offset with respect to one another to better accommodate the different lengths of the toes. As indicated above, Morton's neuroma most typically occurs as the nerve passes under the ligament connecting the metatarsals. Because the location of this point between each set of toes varies based on the length of the toes, gel structures 301a, 301b can be offset to ensure that they will each be positioned appropriately underneath this point.

As shown in FIGS. 3A and 3B, gel structures 301a, 301b may have the same height in some embodiments. However, in other embodiments, one gel structure 301a, 301b may have a greater height than the other. As described above, gel structures 301a and 301b can be sandwiched between adhesive materials 302 and 303. In some embodiments, the perimeter of adhesive materials 302 and 303 may be shaped in accordance with the offset orientation of gel structures 301a, 301b as is best shown in FIGS. 3C and 3D.

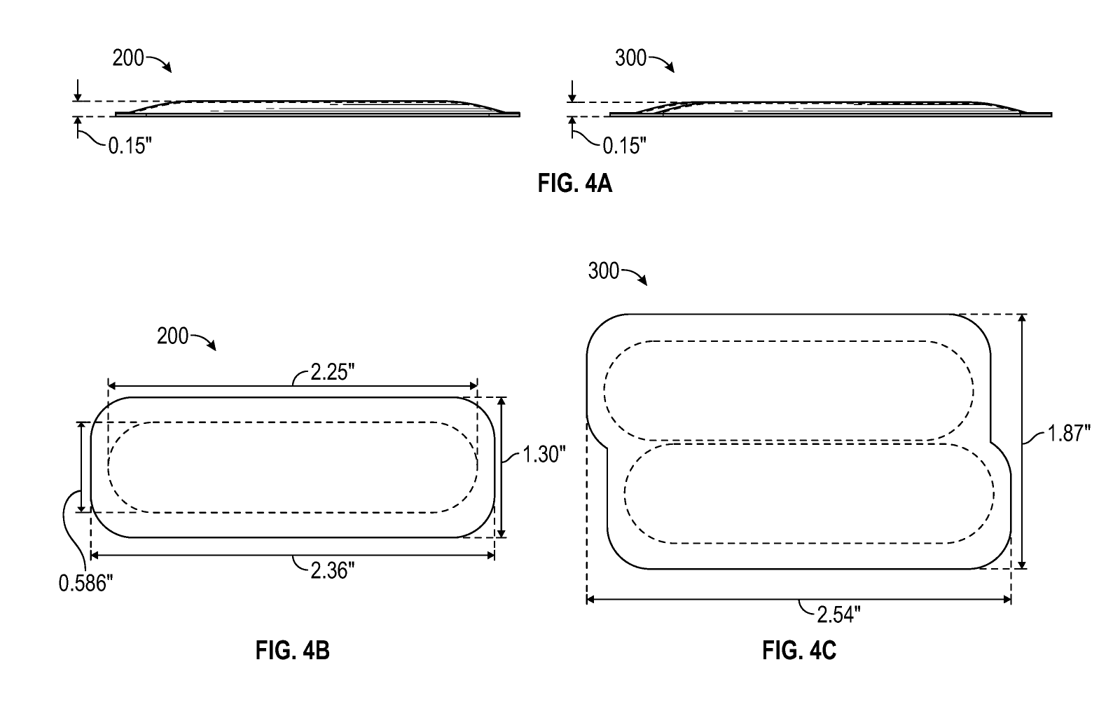

FIGS. 4A-4C illustrate example dimensions of strips 200 and 300 in accordance with one or more embodiments of the present invention. As shown in FIG. 4A, gel structures 201, 301a, and 301b can preferably have a maximum thickness (i.e., the thickness at the top of the arch shape) of approximately 0.125 inches. However, the maximum thickness of the gel structures may be at least 0.08 inches in some embodiments. Adhesive materials 203, 303 will typically have a thickness of around 0.014 inches, while adhesive materials 202, 302 will typically have a thickness of around 0.007 inches. Adhesive materials 203, 303 can be thicker than adhesive materials 202, 302 to provide greater durability underneath gel structures 201, 301a, and 301b.

With these exemplary dimensions, strips 200 and 300 when worn may preferably have a maximum thickness of approximately 0.15 inches. With this thickness, strips 200 and 300 will provide the appropriate separating force without causing undue discomfort to the wearer. However, gel structures of other thicknesses may also be employed in other embodiments. For example, a strip designed for children or women may have a lesser thickness to account for the typically smaller size and spacing of a child's or woman's metatarsals.

FIGS. 4B and 4C illustrate suitable lengths and widths of the components of strips 200 and 300 respectively. With these lengths and widths, gel structure 201, 301a, and 301b will be positioned appropriately between adjacent metatarsals when worn by a person with an average-sized foot. Although not shown, gel structures 301a and 301b can have the same dimensions as shown for gel structure 201 in FIG. 4B.

Of course, strips having different dimensions may be provided for those having smaller or larger feet. In some embodiments, a minimum length and width of the adhesive materials 202 and 203 may be 2.0 inches and 0.75 inches respectively with the length and width of the gel structure 201 being adjusted accordingly (e.g., a minimum length of 1.7 inches and a minimum width of 0.38 inches) so that the gel structure is centered within and surrounded by the adhesive materials. With regards to strip 300, gel structures 301a and 301b may also have minimum lengths and widths of 1.7 inches and 0.40 inches respectively with adhesive materials 302 and 303 having a corresponding length and width to surround the gel structures.

Of course, adhesive materials 202, 203, 302 and 303 can have any length and width as long as there is sufficient overlap to maintain the gel structure(s) within the adhesive material. In some embodiments, adhesive materials 202, 203, 302 and 303 can be formed of a stretchable material. For example, when adhesive materials 302 and 303 are formed of a stretchable material, gel structures 301a and 301b can be slightly separated from one another to enable greater flexibility in the placement of the gel structures on the underside of the foot.

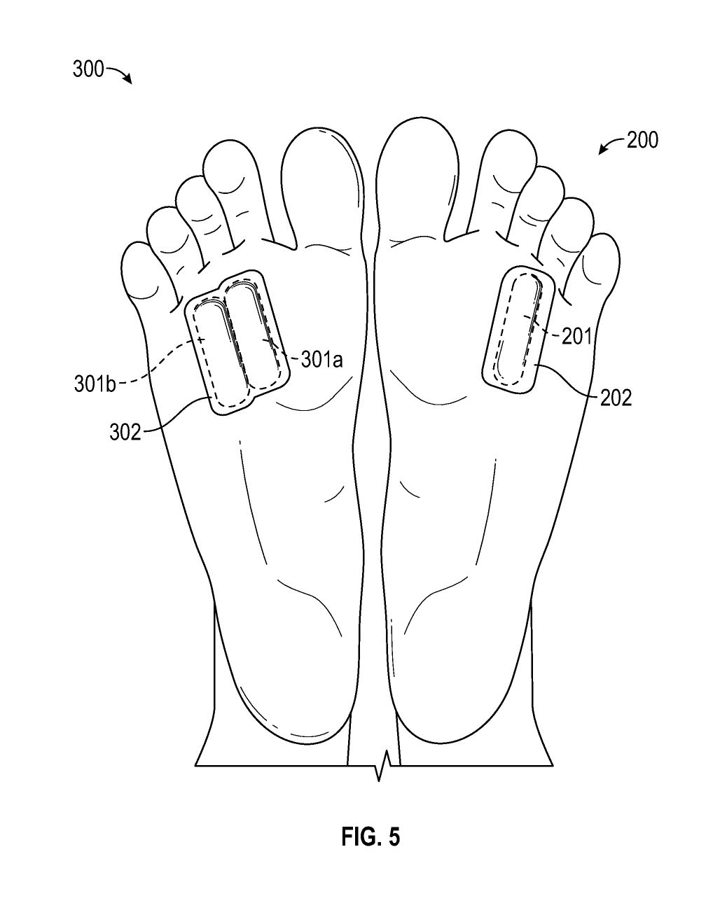

FIG. 5 provides an example of how strips 200 and 300 can be worn. Strip 200 is shown as being worn on the left foot between the third and fourth metatarsals. Similarly, strip 300 is shown as being worn on the right foot with gel structure 301a being positioned between the second and third metatarsals and gel structure 301b being positioned between the third and fourth metatarsals.

It is noted that, in FIG. 5, the lengthwise offset of gel structure 301b is opposite of what is shown in FIGS. 3 and 3A-3D. This represents that a strip having multiple gel structures could be designed specifically for a particular foot. For example, with reference to FIG. 5, strip 300 could be configured as a right foot strip while a strip 300 having gel structure 301a being positioned below gel structure 301b (where below refers to the orientation shown in FIG. 5) could be configured as a left foot strip. In other words, different configurations of strips 300 can be employed so that the wearer can apply a strip having an inside gel structure that is offset towards the tips of the toes from the outside gel structure to thereby account for the inside toes typically being longer than the outside toes.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description.

What is claimed:

1. A pain relief gel strip comprising:
a gel structure having a thickness, a length, and a width; and
an adhesive material having a length and a width that are greater than the length and the width of the gel structure, the gel structure being secured within the length and the width of the adhesive material such that the adhesive material can be employed to secure the gel structure to the underside of the foot.

2. The pain relief gel strip of claim 1, wherein the thickness of the gel structure is approximately 0.15 inches.

3. The pain relief gel strip of claim 1, wherein the gel structure includes a flat bottom surface that is secured to the adhesive material and a top surface that has an arched profile.

4. The pain relief gel strip of claim 1, wherein the width of the gel structure is approximately 0.586 inches.

5. The pain relief gel strip of claim 1, wherein the length of the gel structure approximately 2.25 inches.

6. The pain relief gel strip of claim 1, wherein the gel structure comprises silicone.

7. The pain relief gel strip of claim 1, wherein the adhesive material comprises a top adhesive material and a bottom adhesive material, the gel structure being positioned between the top and bottom adhesive materials.

8. The pain relief gel strip of claim 1, wherein the gel structure and the adhesive material are formed of the same material.

9. The pain relief gel strip of claim 8, wherein the material is silicone.

10. The pain relief gel strip of claim 1, further comprising a second gel structure having a thickness, a length, and a width, the second gel structure also being secured within the length and the width of the adhesive material.

11. The pain relief gel strip of claim 10, wherein the second gel structure is lengthwise offset from the gel structure.

12. The pain relief gel strip of claim 10, wherein the gel structure and the second gel structure have the same thickness.

13. The pain relief gel strip of claim 10, wherein the gel structure and the second gel structure have the same length and the same width.

14. The pain relief gel strip of claim 1, further comprising:
a backing that is secured to the adhesive material.

15. A pain relief gel strip comprising:
a gel structure having a thickness greater than 0.10 inches, a length, and a width; and
an adhesive material having a length and a width that are greater than the length and the width of the gel structure, the gel structure being secured within the length and the width of the adhesive material such that the adhesive material can be employed to secure the gel structure to the underside of the forefoot between adjacent metatarsals, the thickness and the width of the gel structure causing a separating force to be applied on the adjacent metatarsals when weight is placed on the forefoot.

16. The pain relief gel strip of claim 15, wherein the length of the gel structure is at least 1.75 inches, the width of the gel structure is at least 0.38 inches, and the thickness of the gel structure is at least 0.08 inches.

17. The pain relief gel strip of claim 15, wherein the adhesive material comprises a top adhesive material and a bottom adhesive material, the gel structure being positioned between the top and bottom adhesive materials.

18. The pain relief gel strip of claim 15, wherein the gel structure includes a flat bottom surface that is secured to the adhesive material and a top surface that has an arched profile.

19. The pain relief gel strip of claim 15, further comprising:
a second gel structure having a thickness, a length, and a width, the second gel structure also being secured within the length and the width of the adhesive material.

20. A metatarsal pain relief gel strip comprising:
a bottom adhesive material having a width and a length;
a top adhesive material having a width and a length, the top adhesive material having a top side that forms an adhesive surface and a bottom side that is secured to a top side of the bottom adhesive material; and
a gel structure having a thickness, a width, and a height, the width and the length of the gel structure being less than the widths and the lengths of the top and bottom adhesive materials, the gel structure being secured between the top and bottom adhesive materials, the gel structure having a generally flat bottom side that faces the top side of the bottom adhesive material and a top side that protrudes upwardly away from the top side of the bottom adhesive material, the top side of the gel structure having an arched profile;
wherein the top side of the adhesive material is configured to be secured to the underside of the forefoot to thereby maintain the gel structure between adjacent metatarsals with the top side of the gel structure protruding between the metatarsals to thereby apply a separating force against the metatarsals when weight is placed on the forefoot.
